(19) United States
(12) Patent Application Publication
(10) Pub. No.: US 2016/0317807 A1
(43) Pub. Date: Nov. 3, 2016

(54) CLOSED-LOOP BRAIN STIMULATION APPARATUS AND METHOD FOR GENERATING STIMULATION VOLTAGE THEREOF

(71) Applicant: National Tsing Hua University, Hsinchu City (TW)

(72) Inventors: Hsi-Pin Ma, Hsinchu City (TW); Hung-Chih Chiu, Hsinchu City (TW)

(21) Appl. No.: 14/749,656

(22) Filed: Jun. 25, 2015

(30) Foreign Application Priority Data
Apr. 28, 2015 (TW) 104113520

(51) Int. Cl. A61N 1/36; A61N 1/02; A61B 5/04; A61N 1/18; A61B 5/00

(52) U.S. Cl. CPC A61N 1/36; A61N 1/18; A61B 5/4076; A61B 5/04001; A61N 1/025

(57) ABSTRACT

A closed-loop brain stimulation apparatus and a method for generating a stimulation voltage thereof are provided. The closed-loop brain stimulation apparatus includes a brain signal receiving apparatus, a controller and a stimulation voltage generator. The brain signal receiving apparatus receives a plurality of brain signals. The controller performs phase correlation operations on the brain signals during a plurality continuous time periods respectively for obtaining a plurality of phase operation values, and generates a stimulation enable signal according to the phase operation values. The stimulation voltage generator generates a stimulation voltage according to the stimulation enable signal, and transports the stimulation voltage to an electrode pair.

### CROSS-REFERENCE TO RELATED APPLICATION

This application claims the priority benefit of Taiwan application serial no. 104113520, filed on Apr. 28, 2015. The entirety of the above-mentioned patent application is hereby incorporated by reference herein and made a part of this specification.

### BACKGROUND OF THE INVENTION

1. Field of the Invention

The invention relates to a closed-loop brain stimulation apparatus and a method for generating a stimulation voltage thereof, and relates particularly to a closed-loop brain stimulation apparatus which performs analysis based on a phase characteristic of a brain signal and a method for generating a stimulation voltage thereof.

2. Description of Related Art

In modern medical technology, there have been many techniques relating to a nervous system treatment that have been proposed. In the medical treatments relating to a disease of the nervous system, for example, epilepsy and Parkinson's disease, an application involving an electrical stimulation device is commonly seen.

The electrical stimulation devices of conventional art mainly apply a fast Fourier transform to calculate the power of a particular band of the brain signal, and adapt the power obtained to act as a reference for whether to enable brain stimulation. However, the Fourier transform is adapted for processing a calm and steady signal; and the spectral analysis that is performed on the brain signal which has instantaneous changes yield poor results.

In addition, in conventional art, it is necessary to select an analysis window of different sizes when performing analysis of the brain signals. Therefore, this results in a difference in the after analysis power spectral density. Thus, in conventional art, it is necessary to quantify the energy. As a result, many small energy signals disappear due to the quantifying process causing a severe decrease in the sensitivity of the analyzed signal.

Furthermore, because brain signals are typically obtained through a vast amount of nerve signals, therefore only using a spectral analysis method as the reference for enabling the stimulation voltage is too simple for the combination of a multi-layered structure of a neural network.

### SUMMARY OF THE INVENTION

The invention provides a closed-loop brain stimulation apparatus and a method for generating a stimulation voltage thereof, effectively enhancing the accuracy of the brain signal analysis and enhancing the effect of brain stimulation.

The closed-loop brain stimulation apparatus of the invention includes a brain signal receiving apparatus, a controller and a stimulation voltage generator. The brain signal receiving apparatus receives a plurality of brain signals. The controller is coupled with the brain signal receiving apparatus and performs phase correlation operations on the brain signals during a plurality of continuous time periods respectively for obtaining a plurality of phase operation values, and generating a stimulation enable signal according to each phase operation value. The stimulation voltage generator is coupled with the controller, and generates a stimulation voltage according to the stimulation enable signal and transports the stimulation voltage to an electrode pair.

In an embodiment of the invention, the stimulation enable signal is generated when the controller determines an absolute value of each phase correlation operation is not smaller than a preset phase threshold value.

In an embodiment of the invention, the controller sets the preset phase threshold value according to a plurality of reference data values.

In an embodiment of the invention, the closed-loop brain stimulation apparatus further includes a memory device. The memory device is coupled with the controller, and adapted to store the reference data values.

In an embodiment of the invention, a plurality of probes are coupled with the brain signal receiving apparatus, and receives the brain signals, wherein the probes are contacted to a plurality of regions of a tested body.

In an embodiment of the invention, the controller further comprises a plurality of power spectral densities of the brain signals calculated during the continuous time periods respectively, and the stimulation enable signal is generated according to each power spectral density and the corresponding phase operation value.

In an embodiment of the invention, the stimulation enable signal is generated when the controller determines each power spectral density is not smaller than a preset power threshold value and the corresponding phase operation value is not smaller than a preset phase threshold value.

In an embodiment of the invention, the closed-loop brain stimulation apparatus further includes a communication unit. The communication unit is coupled with the controller, wherein the controller performs data transfers with an external computer through the communication unit.

The method for generating a stimulation voltage of a closed-loop brain stimulation apparatus, includes receiving a plurality of brain signals. Performing phase correlation operations on the brain signals during a plurality of continuous time periods respectively for obtaining a plurality of phase operation values, and generating a stimulation enable signal according to each phase operation value. Generating a stimulation voltage according to the stimulation enable signal and transporting the stimulation voltage to an electrode pair.

Based on the above, the invention uses the phase characteristics of the brain signal as the basis for whether to send out the stimulation voltage. In this way, the stimulation voltage generated will not be affected by the difference in the power spectral density of the window sizes when pedalling Fourier transform operation caused by the inaccuracy in the determination of the brain signal. The brain signal analysis of the invention may be more accurate through the switching the scale of the range of the phase characteristic analysis in a fixed range (0-360 degrees), and the transmitted timing and the effect of the stimulation signal may be enhanced.

The accompanying drawings are included to provide a further understanding of the invention, and are incorporated in and constitute a part of this specification. The drawings illustrate embodiments of the invention and, together with the description, serve to explain the principles of the invention.

### BRIEF DESCRIPTION OF THE DRAWINGS

FIG. 1 is a schematic diagram of a closed-loop brain stimulation apparatus according to an embodiment of the invention.

FIG. 2A is a schematic diagram of a brain signal.

FIG. 2B is a schematic diagram of a plurality of phase operation values according to an embodiment of the invention.

FIG. 3 is a flow diagram of a method for generating a stimulation voltage used for closed-loop brain stimulation according to an embodiment of the invention.

FIG. 4 is a schematic diagram of a method for determining a stimulation enable signal according to another embodiment of the invention.

FIG. 5 is a flow diagram of a method for generating a stimulation voltage adapted for closed-loop brain stimulation according to another embodiment of the invention.

FIG. 6 is a schematic diagram of a closed-loop brain stimulation apparatus according to another embodiment of the invention.

### DESCRIPTION OF THE EMBODIMENTS

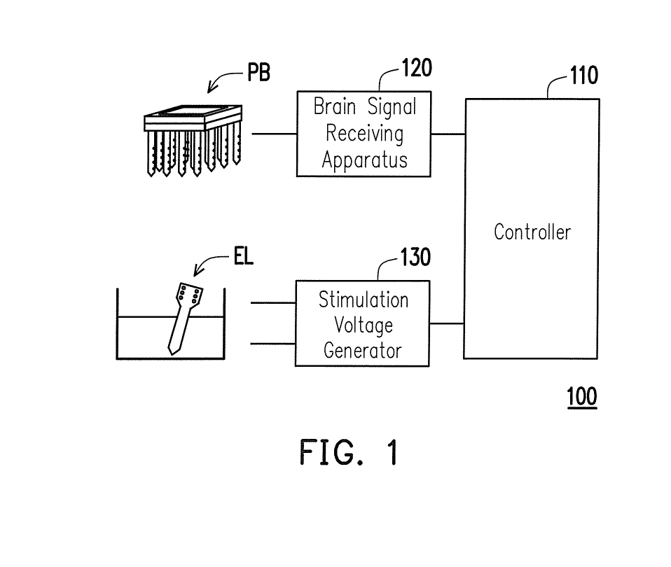

FIG. 1 is a schematic diagram of a closed-loop brain stimulation apparatus according to an embodiment of the invention. Referring to FIG. 1, a closed-loop brain stimulation apparatus 100 includes a controller 110, a brain signal receiving apparatus 120 and a stimulation voltage generator 130. The brain signal receiving apparatus 120 is coupled to a plurality of probes PB and receives a plurality of brain signals through the probes PB which are contacted to the head of a patient. The probes PB may be contacting a plurality of different regions of the head of the patient respectively. The controller 110 is coupled to the brain signal receiving apparatus 120. The controller 110 performs phase correlation operations on the brain signals received in a plurality of continuous time periods so as to obtain a plurality of phase operation values and generates a stimulation enable signal according to each phase operation value.

The controller 110 is also coupled to the stimulation voltage generator 130; and the controller 110 transports the stimulation enable signal to the stimulation voltage generator 130. The stimulation voltage generator 130 may then generate a stimulation voltage according to the stimulation enable signal. The stimulation voltage generator 130 is further coupled to the electrode pair EL and the stimulation voltage is transported to the electrode pair EL. In addition, the stimulation signal may be transported to the brain of the patient by contacting the electrode pair EL with the head of the patient, and thereby performing a treatment.

More specifically, the controller 110 performs phase correlation operations on the brain signals received in order to learn the relationship of the voltage transition states between the brain signals. Namely, the controller 110 may learn a discharge sequence of the nerve signals at different regions through the phase correlation operations. Accordingly, the controller 110 may learn the synchronizing characteristics of the discharge phenomenon of the nerve signals by obtaining the phase operation values through phase correlation operations, and when the discharge synchronizing phenomenon of the nerve signals tend to be severe, the stimulation voltage is generated to disturb the synchronizing discharge state of the nerve signal of the patient's brain, and then achieving a treatment effect.

More specifically, the controller 110 may calculate the absolute value of each phase operation value in each corresponding time period and determine whether the absolute value of each phase operation value is not smaller than a preset phase threshold value for determining whether to generate the stimulation enable signal. When the controller 110 determines the absolute value of each phase operation value is not smaller than the preset phase threshold value, the controller 110 generates the stimulation enable signal. On the other hand, when the controller 110 determines the absolute value of each phase operation value is smaller than the preset phase threshold value, the controller does not generate the stimulation enable signal.

When the controller 110 determines that the stimulation enable signal needs to be generated, the controller 110 may set a stimulation signal switch on flag to 1. On the other hand, when the controller 110 determines the stimulation enable signal does not need to be generated, the controller may set the stimulation signal switch on flag to 0. In this way, the stimulation voltage generator 130 may determine whether to generate the stimulation voltage by determining whether the stimulation signal switch on flag is 1 or 0.

It should be noted, the stimulation voltage provided by the stimulation voltage generator 130 may be performed by a pulse signal. The stimulation voltage generator 130 may generate the stimulation voltage by setting the pulse width and the peak voltage of the pulse signal.

It is worth to note, because the brain signal receiving apparatus 120 continuously obtains the brain signals along with time, therefore the controller 110 may perform phase correlation operations of the brain signals in every time period in a timely manner. The duration of the above mentioned time periods may be set according to the operation speed of the controller 110, such that the controller 110 may have the ability to perform detection actions of the brain signals in a timely manner. That is to say, there are no particular restrictions on the duration of the time periods. It is worth to note, a single time period may include a plurality of brain signal cycles.

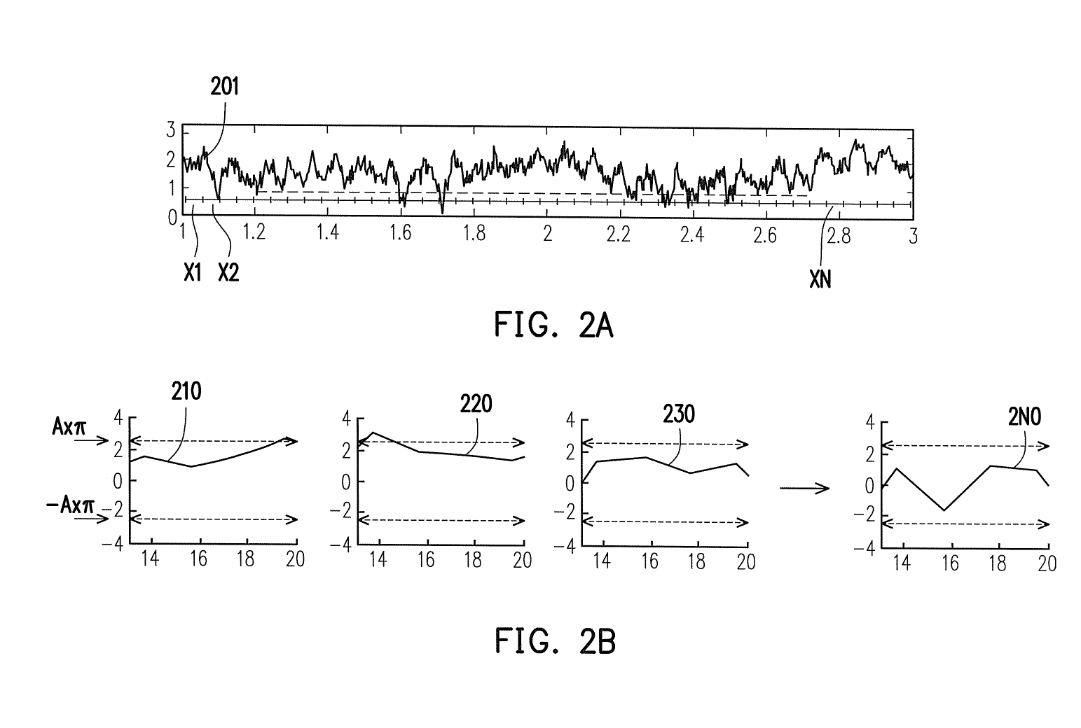

FIG. 2A is a schematic diagram of a brain signal. FIG. 2B is a schematic diagram of a plurality of phase operation values according to an embodiment of the invention. In FIG. 2A, the horizontal axis is time and the vertical axis is amplitude. In FIG. 2B, the horizontal axis is frequency and the vertical axis is the angle. Referring to FIG. 1, FIG. 2A and FIG. 2B, the controller 110 may perform processing on the brain signal 201 sequentially according to the plurality of time periods X1˜XN. A plurality of curves 210-2N0 formed by the phase operation values may be obtained sequentially through executing the phase correlation operations on each time period. The phase operation values may be the absolute value of the result of the phase correlation operation. In the present embodiment, the preset phase threshold value may be set as a product of A and π wherein A is greater than 0 and smaller than 1.

The controller 110 may perform determinations on the phase operation values obtained in each time period. Using the curve 210 obtained in the time period X1-X2 as an example, there appears a situation where the curve 210 is not smaller than the preset phase threshold value A×π, signifying that the synchronizing discharge phenomenon of the brain signal at that moment tends to be severe. Correspondingly, the controller 110 generates the stimulation enable signal such that the stimulation voltage generator 130 generates the stimulation voltage.

In the same way, when in another time period, similarly there appears a situation where the curve 220 is not smaller than the preset phase threshold value A×π, the controller 110 may correspondingly generate the stimulation enable signal such that the stimulation voltage generator 130 generates the stimulation voltage. On the other hand, the curves 230, 2N0 maintain a situation smaller than the preset phase threshold value A×π, therefore during the time period corresponding to the curves 210, 2N0, the controller does not need to generate the stimulation enable signal to drive the generation of the stimulation voltage.

Here, settings for the size of the value A in the preset phase threshold value A×π, may be generated through a plurality of reference data values. More specifically, when the closed-loop brain stimulation apparatus 100 performs treatment on an epilepsy patient, the phase operation values of a plurality of epilepsy patients may be recorded, thereby obtaining reference data values. In this way, the controller 110 may calculate the value of A according to the reference data values to prevent epileptic seizures in a timely manner. Of course, the value of A may be set so as to target a particular patient more appropriately. For example, the controller 110 may perform adjustments to the value of A through monitoring the actual illness state of the patient, so as to optimize the settings of the closed-loop brain stimulation apparatus 100.

The above mentioned reference data values may be stored in any form of memory device, for example, non-volatile memory, hard disk, solid state disk, CD or other memory device known to a person skilled in the art.

It should be noted, the phase operation values in the present embodiment of the invention will not be greater than π. Therefore, the controller 110 may determine when the phase operation value is smaller than or equal to π, and the stimulation enable signal is generated when not smaller than A×π. In addition, in the present embodiment, A may be equal to 0.7.

As may be seen from the above description, the embodiment of the invention may detect the discharge sequence of different brain regions effectively by performing analysis of the brain signals through the phase characteristics, and generate the stimulation voltage according to the discharge sequence, performing treatment on patients even more effectively.

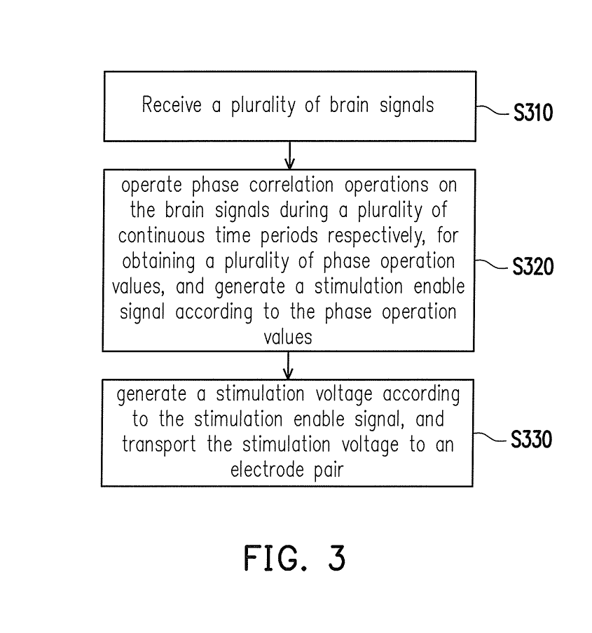

FIG. 3 is a flow diagram of a method for generating a stimulation voltage used for closed-loop brain stimulation according to an embodiment of the invention. Referring to FIG. 3, in a step 310 a plurality of brain signals are received. Step S320 operates phase correlation operations on the brain signals during a plurality of continuous time periods respectively, for obtaining a plurality of phase operation values, and generates a stimulation enable signal according to the phase operation values. Step S330 generates a stimulation voltage according to the stimulation enable signal, and transports the stimulation voltage to an electrode pair.

The details regarding each of the above steps of the embodiment have been described in detail in the aforementioned embodiment and will not be repeated here.

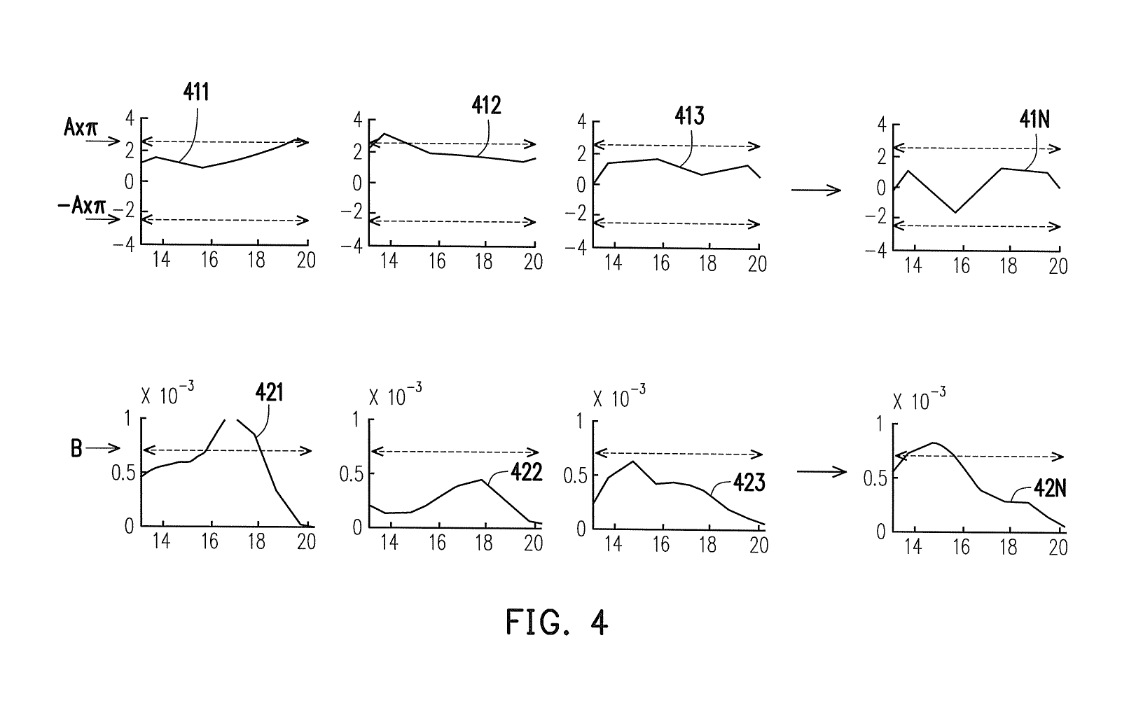

It should be noted, in other embodiments of the invention, the controller 110 aside from determining whether to generate the stimulation enable signal according to the phase operation values obtained by the phase correlation operations, may be further arranged with determining the power spectral density of the brain signal for determining whether to generate the stimulation enable signal. FIG. 4 is a schematic diagram of a method for determining a stimulation enable signal according to another embodiment of the invention. Referring to FIG. 1 and FIG. 4, in FIG. 4, the power spectral density curves 421˜42N and the phase operation value curves 411˜41N corresponding to different periods may be obtained from the brain signals corresponding to different time periods, wherein the power spectral density curves 421˜42N correspond to the phase operation value curves 411˜41N respectively.

The controller 110 may determine whether the power spectral density curve is not smaller than a preset power threshold value and whether the phase operation value curve is not smaller than a preset phase threshold value during the same corresponding time period. When the power spectral density curve is not smaller than the preset power threshold value and the operation value curve is not smaller than the preset phase threshold value, the controller 110 generates the stimulation enable signal through the controller 110. On the other hand, the controller 110 may not generate the stimulation enable signal, when the power spectral density curve is greater than the preset power threshold value, or the phase operation value curve is greater than the preset phase threshold value, or when the power spectral density curve is greater than the preset power threshold value and the phase operation value curve is greater than the preset phase threshold value. That is to say, in FIG. 4, there appears a situation where the power spectral density curve 421 is greater than the preset power threshold value B, and there appears a situation where the corresponding phase operation value curve 411 is greater than the preset phase threshold value A×π, therefore the controller generates the stimulation enable signal. In another time period, there appears a situation in the power spectral density curve 422 that is greater than the preset power threshold value B, and there does not appear a situation in the corresponding phase operation value curve 412 greater than the preset phase threshold value A×π, therefore the controller stops generating the stimulation enable signal.

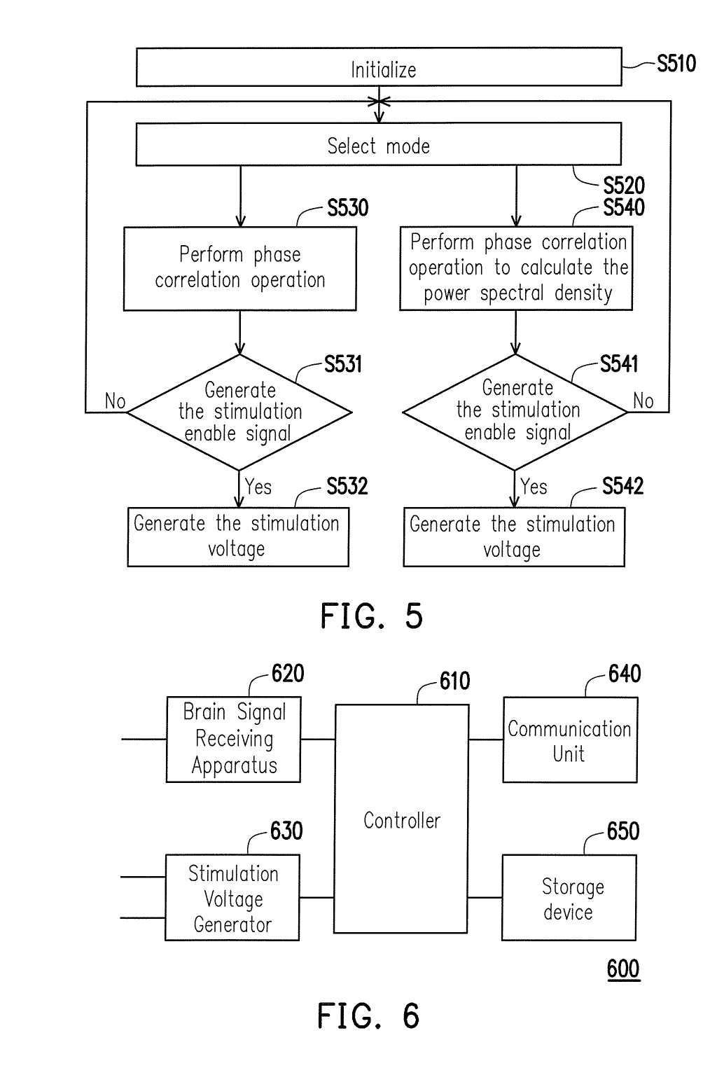

FIG. 5 is a flow diagram of a method for generating a stimulation voltage adapted for closed-loop brain stimulation according to another embodiment of the invention. Referring to FIG. 5, in a step S510, the closed-loop brain stimulation apparatus is initialized. Next, in step S520, the mode is selected. In the step S520, the determination process to be performed on the brain signals may be selected, wherein under the instance where mode 1 is selected, step S530 may be executed. On the other hand, under the instance where mode 2 is selected, step S540 may be executed.

In the step S530, a fast Fourier transform may be performed on the brain signals in each of the time periods, so as to convert the brain signals to the frequency domain for performing analysis. Phase correlation operations are then executed on the brain signals of the frequency domain, and in this way the phase operation values are obtained. Here, the phase correlation operations may be performed using phase correlation operations adapted by one skilled in the art, for example, Spearman's correlation operation. Then, step S531 determines whether to generate the stimulation enable signal according to the determination of whether the phase operation value is not smaller than the preset phase threshold value. When step S531 determines the stimulation enable signal needs to be generated, then step S532 is executed to generate the stimulation voltage according to the stimulation enable signal generated. If step S531 determines the stimulation enable signal does not need to be generated, then return to step S520.

Step S540 performs fast Fourier transform on the brain signals of each time period, so as to convert the brain signals to the frequency domain for performing analysis. Phase correlation operations are then executed on the brain signals of the frequency domain, and in this way the phase operation values are obtained, and the power spectral density of the brain signals are calculated. Next, step S541 determines whether to generate the stimulation enable signal according to the determination of whether the phase operation value is not smaller than the preset phase threshold value and whether the power spectral density is not smaller than the preset power threshold value. When step S541 determines the stimulation enable signal needs to be generated, then step S542 is executed to generate the stimulation voltage according to the stimulation enable signal generated. If step S541 determines the stimulation enable signal does not need to be generated, then return to step S520.

FIG. 6 is a schematic diagram of a closed-loop brain stimulation apparatus according to another embodiment of the invention. Referring to FIG. 6, a closed-loop brain stimulation apparatus 600 includes a controller 610, a brain signal receiving apparatus 620, a stimulation voltage generator 630, a communication unit 640 and a storage device 650. The difference between the embodiment of FIG. 1 lies in, the controller 610 of the closed-loop brain stimulation apparatus 600 is additionally coupled to the communication unit 640 and the storage device 650. The storage device 650 is adapted to store reference data values, in which the reference data values may be provided to the controller 610 as a basis for setting the preset phase threshold value. In addition, the communication unit 640 may act as a medium for the controller 610 for performing communications with an external computer, wherein the external computer may be various kinds of electronic devices, for example, a desktop computer, a notebook computer, a cell phone, a tablet PC, a smart TV and the like.

The communication unit 640 may perform wired data transfers, wireless data transfers or shared wired and wireless data transfers. The external computer may obtain various information obtained by the closed-loop brain stimulation apparatus 600 through the communication unit 640. The external computer may also transport data to the closed-loop brain stimulation apparatus 600 through the communication unit 640, or give instructions. For example, in the mode selection process in the embodiment of FIG. 5, mode selection may be performed by the external computer through the communication unit 640.

In summary, the invention uses the phase characteristics of the brain signal as the basis for whether to send out the stimulation voltage. The state of the synchronizing discharge of the nerve signal is monitored by the phase correlation operations and at the most suitable time the stimulation voltage is provided for performing treatment. In this way, small variations in the brain signal may be monitored effectively; the sensitivity of the brain signal detection may be enhanced; and the effectiveness of the system may be fully enhanced.

What is claimed is:

1. A closed-loop brain stimulation apparatus, comprising:
a brain signal receiving apparatus, receiving a plurality of brain signals;
a controller, coupled with the brain signal receiving apparatus, and performing phase correlation operations on the brain signals during a plurality of continuous time periods respectively for obtaining a plurality of phase operation values, and generating a stimulation enable signal according to each phase operation value; and
a stimulation voltage generator, coupled with the controller, and generating a stimulation voltage according to the stimulation enable signal and transporting the stimulation voltage to an electrode pair.

2. The closed-loop brain stimulation apparatus as claimed in claim 1, wherein, the stimulation enable signal is generated when the controller determines an absolute value of each phase correlation operation is not smaller than a preset phase threshold value.

3. The closed-loop brain stimulation apparatus as claimed in claim 1, wherein the controller sets the preset phase threshold value according to a plurality of reference data values.

4. The closed-loop brain stimulation apparatus as claimed in claim 3, further comprising:
a memory device, coupled with the controller, and adapted to store the reference data values.

5. The closed-loop brain stimulation apparatus as claimed in claim 3, further comprising a plurality of probes coupled with the brain signal receiving apparatus, and receiving the brain signals, wherein the probes are contacted to a plurality of regions of a test body.

6. The closed-loop brain stimulation apparatus as claimed in claim 1, wherein the controller further comprises a plurality of power spectral densities of the brain signals calculated during the continuous time periods respectively, and the stimulation enable signal is generated according to each power spectral density and the corresponding phase operation value.

7. The closed-loop brain stimulation apparatus as claimed in claim 6, wherein the stimulation enable signal is generated when the controller determines each power spectral density is not smaller than a preset power threshold value and the corresponding phase operation value is not smaller than a preset phase threshold value.

8. The closed-loop brain stimulation apparatus as claimed in claim 6, further comprising:
a communication unit, coupled with the controller,
wherein the controller performs data transfers with an external computer through the communication unit.

9. A method for generating a stimulation voltage of a closed-loop brain stimulation apparatus, comprising:
receiving a plurality of brain signals;
performing phase correlation operations on the brain signals during a plurality of continuous time periods respectively for obtaining a plurality of phase operation values, and generating a stimulation enable signal according to each phase operation value; and
generating a stimulation voltage according to the stimulation enable signal and transporting the stimulation voltage to an electrode pair.

10. The method for generating the stimulation voltage as claimed in claim 9, wherein the step of generating the stimulation enable signal according to each phase operation value comprises:
generating the stimulation enable signal when determining an absolute value of each phase operation value is not smaller than a preset phase threshold value.

11. The method for generating the stimulation voltage as claimed in claim 9, further comprising:
calculating a plurality of power spectral densities of the brain signals during the continuous time periods respectively, and generating the stimulation enable signal according to each power spectral density and the corresponding phase operation value.

12. The method for generating the stimulation voltage as claimed in claim 11, wherein the step of generating the stimulation enable signal according to each power spectral density and the corresponding phase operation value comprises:
generating the stimulation enable signal when determining each power spectral density is not smaller than a preset power threshold value and the corresponding phase operation value is not smaller than a preset phase threshold value.
